(19) United States
(12) Patent Application Publication
(10) Pub. No.: US 2008/0096233 A1
(43) Pub. Date: Apr. 24, 2008

(54) Isolation Of Immune Complexes

(76) Inventors: Karla M. Robotti, Mountain View, CA (US); Magdalena A. Bynum, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/551,321

(22) Filed: Oct. 20, 2006

(51) Int. Cl. G01N 33/574; G01N 33/554; G01N 33/558

(52) U.S. Cl. 435/7.23; 436/514; 435/7.32

(57) ABSTRACT

The present invention provides methods of detecting and/or isolating immune complexes. The antigen in the immune complexes can further be isolated from the immune complexes to facilitate research and characterization of the antigen. Other embodiments of the present invention can be used to, inter alia, develop assays for detecting immune complexes, diagnose diseases or medical conditions, as well as determine the correlation between the presence of immune complexes and diseases and medical conditions of interest. Still other embodiments provide reagents, equipments and kits for implementing the methods.

### BACKGROUND OF THE INVENTION

One way the immune system responds to a foreign matter, or antigen, is to generate specific antibodies that can recognize and bind the antigen to form an antigen-antibody complex, or immune complex (IC). The formation of immune complexes facilitates neutralization and/or removal of the antigen.

Immune complexes are normally not found in the circulation. However, if immune complexes are formed faster than they can be cleared, immune complexes can appear in the blood, known as circulating immune complexes (CICs). Immune complexes can also become localized in vascular walls, particularly in renal glomerular capillaries. A variety of pathological disorders have been associated with the circulation of immune complexes or their deposition into tissues. These disorders include autoimmune diseases, such as rheumatoid arthritis and systemic lupus erythematosus (SLE); neoplastic diseases, such as leukemia, ovarian cancer and breast cancer; infectious diseases due to bacteria, viruses or parasites; and diseases that do not belong to any of the above categories. Both exogenous and endogenous antigens can trigger pathogenic immune responses, resulting in immune complex disorders. Thus, to diagnose and/or study the pathogenesis of these disorders, it is important to detect immune complexes or to identify the antigen therein.

Several approaches have been used to detect immune complexes in serum or tissues. The existence of tissue-bound immune complexes has been revealed mainly by immunohistological techniques (Freedman et al., 1962; Paronetto, et al., 1965). The antigen in an immune complex, if known, can serve as the basis of detection. For instance, hepatitis B antigen-antibody complexes were detected by using electronmicroscopy (Almeida et al., 1969); detection of lactic dehydrogenase virus (LDV)-antibody complexes was achieved in mice by the demonstration of a decreased infectivity of LDV in serum after precipitation of the circulating immunoglobulins (Notkins et al., 1966). Whether or not the antigen is known, detection of circulating immune complexes can be accomplished by procedures that take advantage of the great molecular weight of immune complexes, such as chromatography, gel filtration, electrophoresis and ultracentrifugation in sucrose density gradients. Polyethelene glycol (PEG), which can be used to fractionate plasma proteins according to their sizes, is commonly employed at low concentrations to precipitate immune complexes while free antigens and antibodies remain soluble. The complement-fixing properties of immune complexes have also been utilized. Since C1q is the first component of the complement system to bind an immune complex in a complement cascade, anti-C1q antibodies have been used to detect or measure immune complexes.

These approaches, however, are either time-consuming or expensive. Therefore, it is desirable to develop fast, efficient and/or less costly methods to detect immune complexes.

### SUMMARY OF THE INVENTION

The present invention provides methods of detecting and/or isolating immune complexes. The antigen in the immune complexes can further be isolated from the immune complexes to facilitate research and characterization of the antigen.

Some embodiments of the present invention provides a method for isolating an antigen from an immune complex, comprising:
(a) applying a fluid sample to a first column that comprises an immunoglobulin-binding agent;
(b) collecting the flow-through eluant and detecting if immune complexes are present in the eluant;
(c) if immune complexes are present in the flow-through eluant of step (b), dissociating the immune complexes and isolating the antigen;
(d) if immune complexes are not present in the flow-through eluant of step (b):
   (d1) eluting the first column with an elution buffer that dissociates immune complexes;
   (d2) applying the eluant of step (d1) to a second column comprising the immunoglobulin-binding agent;
   (d3) collecting the flow-through eluant of step (d2).

Thus, step (c) or (d) is optional, depending on the outcome of step (b). Furthermore, if it is already known that the immune complex would appear in the flow-through, it is not necessary to detect immune complexes in step (b). Instead, the flow-through eluant is collected and step (c) is performed. Conversely, if it is already known that the immune complex would not appear in the flow-through, both steps (b) and (c) can be omitted.

The immunoglobulin-binding agent is capable of binding an immunoglobulin selected from the group consisting of IgM, IgG, IgA, IgE and IgD. Preferably, it binds to IgG.

The first or second column may comprise an agent capable of binding at least one molecule selected from the group consisting of other immunoglobulins, albumin, transferrin, haptoglobin, antitrypsin, and fibrinogen. In some preferred embodiments, the first or second column comprises agents capable of binding IgG, IgA, albumin, transferrin, haptoglobin, and antitrypsin.

The binding agents, including the immunoglobulin-binding agent, can be any agents known in the art. Preferably, the binding agent is an antibody.

The first or second column may be in any form or size. In some embodiments, the column is a spin column.

The fluid sample is preferably serum, plasma, cerebrospinal fluid, ascites fluid, peritoneal fluid, or synovial fluid. More preferably, the sample is serum or synovial fluid.

In some embodiments, the fluid sample is obtained from a subject suffering from or suspected of having a disease or medical condition selected from the group consisting of rheumatoid arthritis, systemic lupus erythematosus, autoimmune pancreatitis, diabetes, hepatitis B, Lyme disease, pulmonary tuberculosis, lung cancer, leukemia (particularly acute myelogenous leukemia), ovarian cancer and breast cancer.

Another aspect of the present invention provides methods of developing an assay for the detection of immune complexes, particularly circulating immune complexes, in a fluid sample from a subject having a disease or medical condition. Thus, in some embodiments, a fluid sample is applied to a first column comprising an immunoglobulin-binding agent, and the flow-through eluant is examined for the presence of immune complexes. If immune complexes are found in the flow-through, this method can be used to partially purify immune complexes from fluid samples from other subjects having the same disease or medical condition, thereby facilitating detection of immune complexes. If immune complexes are not found in the flow-through, they can be eluted from the column according to methods disclosed herein, again facilitating detection of immune complexes.

Once this assay is developed, it can be used to diagnose the disease or medical condition. Thus, yet another aspect of the present invention provides methods of diagnosing a disease or medical condition, comprising applying a fluid sample from a subject suspected of having the disease or medical condition to a column that comprises an immunoglobulin-binding agent under conditions that allow immune complexes in the sample to bind to the immunoglobulin-binding agent, eluting the column with a solution that would dissociate immune complexes. If the eluate contains immune complexes or the underlying antigen, the subject can be diagnosed as having the disease or medical condition. Alternatively, the sample is applied to a column that comprises an immunoglobulin-binding agent under conditions wherein immune complexes in the sample do not bind to the immunoglobulin-binding agent, and collect the flow-through eluate from the column. If the eluate contains immune complexes, the subject can be diagnosed as having the disease or medical condition.

Similarly, yet another aspect of the present invention provides methods of determining whether a disease or medical condition is associated with the presence of immune complexes. For this purpose, fluid samples are obtained from subjects known to have the disease or medical condition. Control samples are also collected from subjects who are known not to have the disease or medical condition. All the samples are individually tested for the presence of immune complexes according to the methods described herein, and an statistical analysis is performed to determine the correlation between the disease state and the presence of immune complexes. In these embodiments, serum samples are preferably used to detect circulating immune complexes and their association with the disease or medical condition, but other fluid samples can also be utilized.

Reagents, equipments and kits useful to implement various embodiments of this invention are also provided. For example, a kit may comprise a column that contains an immunoglobulin-binding agent, and reagents for detecting immune complexes.

The details of one or more embodiments of the invention are set forth in the description below. Other features, objects, and advantages of the invention will be apparent from the description and from the claims.

It should be noted that, as used herein, the singular forms “a” and “an” include their plural counterparts unless the context clearly dictates otherwise. For example, a reference to “an immunoglobulin-binding agent” includes combinations of immunoglobulin-binding agents which may or may not be identical.

### DETAILED DESCRIPTION

The present invention provides methods of detecting and/or isolating immune complexes. The antigen in the immune complexes can further be isolated from the immune complexes to facilitate research and characterization of the antigen. Prior to describing the invention in further detail, the terms used in this application are defined as follows unless otherwise indicated.

### DEFINITION

To “isolate” an antigen from an immune complex means to separate the antigen from the immune complex such that the antigen is not associated with the antibody or other components of the immune complex. In a mixture comprising multiple immune complexes, wherein all the immune complexes are either identical or not identical, an antigen may be isolated from its corresponding immune complex while the antigens in the other immune complexes in the mixture remain un-isolated. The purity of isolated antigens may be, for example, at least about 10, 20, 30, 40, 50, 60, 70, 80, 90, 95, 98, 99 or 99.9% (as determined by dividing the amount of the isolated antigen by the amount of total protein in the solution or mixture comprising the isolated antigen).

Similarly, to “isolate” an immune complex from a sample means to separate the immune complex from at least one other component in the sample. Preferably, after the isolation, the purity of the immune complex is at least about 10, 20, 30, 40, 50, 60, 70, 80, 90, 95, 98, 99 or 99.9% (as determined by dividing the amount of the immune complex by the amount of total protein in the solution or mixture comprising the immune complex).

An “immune complex” is a complex comprising at least one antigen and at least one antibody, wherein the antigen and antibody are associated by non-covalent linking. The immune complex may comprise other components, such as complement factors, particularly C1q.

An “immunoglobulin-binding agent” is a substance that specifically binds an immunoglobulin (i.e., an IgM, IgG, IgA, IgE or IgD). The immunoglobulin-binding agent should bind to a region of the immunoglobulin that is common to all the immunoglobulin in the same class (i.e., IgM, IgG, IgA, IgE or IgD) or subclass (e.g., IgG1, IgG2, IgG3 or IgG4). The immunoglobulin-binding agent can be any agent that is available in the art, including but not limited to antibodies, Protein A, Protein G, Protein L, and engineered proteins such as those described in U.S. Pat. No. 5,831,012.

As used herein, an “antibody” that is useful in this invention as a binding agent includes, without being limited to, a monoclonal antibody, polyclonal antibody, multi-specific antibody, chimeric antibody, humanized antibody, antibody fragment (such as an Fab, Fab′, F(ab′)₂, or Fv fragment), diabody, triabody, tetrabody, linear antibody, single-chain antibody, and other engineered antibodies established in the art.

A “fluid sample” is a sample of which at least 50% (by weight) is in the liquid state. Preferably, at least 60, 70, 80, 85, 90, 95, 96, 97, 98, 99, 99.5, or 99.9% of the fluid sample is in the liquid state. Exemplary fluid samples include serum, plasma, cerebrospinal fluid, ascites fluid, peritoneal fluid, and synovial fluid.

As used herein, a “subject” is a mammal, particularly a mammal selected from the group consisting of human, mouse, rat, rabbit, dog, cat, and non-human primate.

To “diagnose” a disease or medical condition in a subject refers to determining whether the subject has the disease or medical condition, or determining the severity or stage of the disease or medical condition.

Identification of the Antigen in an Immune Complex

The present invention provides methods of isolating immune complexes, and the antigens therein, by using a double-affinity column procedure. In some embodiments of the present invention, a fluid sample is applied to a first affinity column comprising an immunoglobulin-binding agent. The flow-through eluant is collected and examined for the presence of immune complexes. If immune complexes are present in the flow-through, the flow-through can be used to further isolate and characterize the antigen. If immune complexes are not present in the flow-through, the column is eluted with a buffer that can dissociate antigen-antibody complexes. Thus, the antigen is eluted from the column. This eluant is applied to a second column that comprises the same immunoglobulin-binding agent as the first column, and the flow-through, containing the antigen, is collected for the identification and characterization of the antigen.

The presence of immune complexes in the flow-through of the first column or in any other step can be determined by any method established in the art. For example, the eluant can be incubated with [¹²⁵I]C1q first, then with PEG (to a final concentration of 2.5% of PEG). After the incubation, the mixture is centrifuged and the radioactivity in the precipitates measured (see, e.g., Nydegger et al., 1974). Any radioactivity in the PEG precipitates over background would indicate that the eluant contains immune complexes. Another exemplary method is to precipitate immune complexes with PEG, and measure the level of immune complexes by ELISA using, e.g., anti-C1q antibodies. If the eluant is known not to contain free C1q, immune complexes can be directly quantitated with anti-C1q antibodies and ELISA. Alternatively, immune complexes can be detected by an ELISA wherein the first antibody is an anti-C1q antibody bound to an ELISA plate, and the secondary antibody is an anti-IgG antibody (for IgG-containing immune complexes). See, e.g., Catalogue No. RDI-CG59211, Immuno Biological Laboratories, Hamburg, Germany (http://www.research.com/rdikits/cg59211.htm); Tuzun et al, 2004.

Numerous reagents are known in the art for the dissociation of immune complexes, including both acid and alkaline buffers, as well as detergents. For example, reagents such as isotonic citrate at a pH of about 3.2 and EDTA at a pH of about 7.4 are both effective. Other exemplary reagents and methods of use can be found, for example, in Harlow and Lane, 1988. In some embodiments of the present invention, the immune complexes are dissociated under a denaturing condition, and the antigen cannot form a complex with the antibody again after the dissociation. These embodiments are preferred if the antigen is to be applied to a second column that binds the antibody, and the antigen is to be collected from the flow-through of this second column.

Characterization of the antigen can be achieved by well-known analytical methods in the art, such as electrophoresis, gel filtration, liquid chromatography, gas chromatography, mass spectrometry, sequencing, or any combination thereof.

The embodiments described above can be modified for better results based on the properties of the sample. For example, if immune complexes in a serum sample appear in the flow-through in the first column, it is desirable to also include in the first column agents that can bind the major abundant serum proteins, such as albumin, transferring, haptoglobin, antitrypsin, fibronectin and/or other immunoglobins. As a result, more than 90% of the proteins in a serum sample can be removed in one step by the first column, leaving immune complexes in the flow-through. For this purpose, the Multiple Affinity Removal System columns (Agilent Technologies, Santa Clara, Calif.) can be conveniently used. The flow-through can then be analyzed by various methods chosen by the artisan. One option is to dissociate the antigen from the immune complexes and separate the antigen by using a second affinity column that comprises immunoglobulin-binding agents. The antibody would bind to the second column and the flow-through would contain the antigen. This second column may also optionally comprise binding agents for the major abundant serum proteins in order to remove the abundant serum proteins that have flown through the first column, for example, due to overloading of the first column. For all embodiments of the present invention where the first and second columns have the same binding specificities, the first column can optionally be reused as the second column after appropriate reconstitution and re-equilibration.

In circumstances where immune complexes are not found in the flow-through of the first column, it is preferable not to include any binding agents in the first column other than the immunoglobulin-binding agent. On the other hand, the second column may preferably comprise binding agents for other proteins, such as the abundant serum proteins, to separate these proteins from the antigen of interest. It should be noted, however, that in the interest of speed, ease of use, and cost reduction, the artisan may choose to use a column such as the Multiple Affinity Removal System columns as both the first and second columns even if immune complexes bind to the first column.

Thus, in certain preferred embodiments, the Multiple Affinity Removal System columns are used as both the first and second columns. As discussed above, these columns are useful whether the immune complexes appear in the flow-through of the first column or not. Since these columns are pre-packed columns that can be operated in a matter of minutes, antigens in immune complexes can be isolated in a fast and cost-effective manner. In contrast, the commonly used PEG precipitation method for isolating immune complexes takes at least several hours.

Detection of Immune Complexes

The present invention can also be used to detect immune complexes, particularly circulating immune complexes. Since various diseases or medical conditions have been associated with circulating immune complexes, the present invention can be used to diagnose these diseases or medical conditions by the detection of immune complexes.

To detect immune complexes, e.g., for diagnostic purposes, the properties of the immune complexes must be known prior to the detection. In particular, it is necessary to know whether the immune complexes bind to an immunoglobulin-binding agent to be used. This can be determined by the methods disclosed herein. For example, to determine the properties of the immune complex associated with rheumatoid arthritis, serum samples from rheumatoid arthritis patients can be tested in a column that comprises antibodies capable of binding IgG. The flow-through is then tested for the presence of immune complexes. If immune complexes do not appear in the flow-through, the conditions for eluting the immune complexes from the column are then determined (see, e.g., pages 547-552 of Harlow and Lane, 1988).

Thus, in some diagnostic embodiments of this application, a fluid sample from a subject suspected of having a disease or medical condition is applied to a column that comprises an immunoglobulin-binding agent. The flow-through is collected for the detection of the immune complex if it has been pre-determined that the immune complex would appear in the flow-through of this kind of column, and the presence of immune complexes is indicative of the disease or medical condition. If it has been pre-determined that the immune complex is retained in this kind of column, the column is eluted under conditions that dissociate the immune complex from the column, and the eluate is tested for immune complexes or the underlying antigen. The presence of immune complex or the antigen is indicative of the disease or medical condition.

Some other embodiments of this invention provide methods of diagnosing a disease or medical condition in a subject, comprising providing a fluid sample from the subject and arranging for the detection of immune complexes. Immune complexes can be detected, for example, by the procedures described above.

Kits

This invention also provides kits for the isolation or detection of immune complexes or antigens. The kits typically comprise a column that contains an immunoglobulin-binding agent, and reagents for detecting immune complexes (such as PEG or C1q-specific antibodies). Some preferred kit embodiments comprise multiple columns that contain an immunoglobulin-binding agent. The kits may also comprise reagents for running the column(s), including, for example, a loading buffer, elution buffer, washing buffer, and/or equilibration buffer. An instruction of use or container for the components in the kits may also be included.

The following examples are offered to illustrate this invention and are not to be construed in any way as limiting the scope of the present invention. While this invention is particularly shown and described with references to preferred embodiments thereof, it will be understood by those skilled in the art that various changes in form and details may be made therein without departing from the spirit and scope of the invention as defined by the appended claims.

### EXAMPLES

In the example below, the following abbreviations have the following meanings. Abbreviations not defined have their generally accepted meanings.

° C.=degree Celsius

hr=hour

min=minute

sec=second

μM=micromolar

mM=millimolar

M=molar

ml=milliliter

μl=microliter

mg=milligram

μg=microgram

BSA=bovine serum albumin

CIC=circulating immune complex

ELISA=enzyme-linked immunosorbent assay

HRP=horseradish peroxidase

LC=liquid chromatography

MS=mass spectrometry

PBS=phosphate-buffered saline

PEG=polyethelene glycol

### Example 1

Isolation of Immune Complexes from the Serum of SLE Patients

Serum samples from systemic lupus erythematosus (SLE) human patients are collected and pooled. Serum samples from humans not suffering from SLE are also pooled as a normal control. A 15 μl aliquot of each of the pooled samples is diluted with 185 μl of a loading buffer (Buffer A in the Multiple Affinity Removal Kits). This 200 μl of sample is loaded onto an Agilent High Capacity Multiple Affinity Removal Spin Cartridge (PN 5188-5341; Agilent Technologies, Santa Clara, Calif.). The cartridge is immediately spun for 1.5 min at 100×g to load the sample, and any flow-through is collected as flow-through fraction 1. More loading buffer (400 μl Buffer A) is added and the cartridge is again centrifuged for 2.5 min at 100×g. The flow-through is collected in the same tube containing flow-through fraction 1. Another 400 μl of loading buffer is added to the cartridge and the wash step is repeated. Flow-through fraction 1 and the wash are combined (collectively “the flow-through”), optionally concentrated to a small volume, and subjected to an ELISA for the detection of immune complexes as modified from the method described in Tuzun et al., 2004. Briefly, microtiter plates are covered with goat antibodies to human C1q and blocked with 2% BSA. The flow-through (30 μl) is added and incubated at 37° C. for 90 min. After four washes, HRP-conjugated goat anti-human IgG is used as a secondary antibody to detect immune complexes. The results show that the flow-through from the patient pool or normal control contains no detectable levels of immune complexes.

The cartridge is eluted with the elution buffer (Buffer B) provided by the manufacturer by pushing 2 ml of elution buffer through the spin cartridge into a collection tube. The eluant is concentrated, buffer-exchanged if desired, and applied to a fresh Multiple Affinity Removal spin cartridge. The flow-through is collected as described above. The proteins in the flow-through are analyzed by LC-MS, and the profile from the patient pool is compared to that of the normal control. The patient pool has a peak that is not found in the normal control, and this peak is subjected to further analysis.

### REFERENCES CITED

U.S. Pat. No. 5,831,012.

Almeida J D, Waterson A P. Immune complexes in hepatitis. Lancet. 1969 November 8;2(7628):983-986.

Freedman P, Markowitz A S. Gamma Globulin And Complement In The Diseased Kidney. J Clin Invest. 1962 February;41(2):328-334.

Harlow and Lane. Antibodies. A Laboratory Manual. Cold Spring Harbor Laboratory, New York, N.Y., 1988.

Notkins A L, Mahar S, Scheele C, Goffman J. Infectious virus-antibody complex in the blood of chronically infected mice. J Exp Med. 1966 July 1;124(1):81-97.

Nydegger U E, Lambert P H, Gerber H, Miescher P A. Circulating Immune Complexes in the Serum in Systemic Lupus Erythematosus and in Carriers of Hepatitis B Antigen. QUANTITATION BY BINDING TO RADIO LABELED C1q. J Clin Invest. 1974 August; 54(2): 297-309.

Paronetto F, Koffler D. Immunofluorescent localization of immunoglobulins, complement, and fibrinogen in human diseases. I. Systemic lupus erythematosus. J Clin Invest. 1965 October;44(10):1657-1664.

Tuzun E, Scott B G, Yang H, Wu B, Goluszko E, Guigneaux M, Higgs S, Christadoss P. Circulating immune complexes augment severity of antibody-mediated myasthenia gravis in hypogammaglobulinemic RIIIS/J mice. J Immunol. 2004 May 1;172(9):5743-52.

All of the publications, patents and patent applications cited above or elsewhere in this application are herein incorporated by reference in their entirety to the same extent as if the disclosure of each individual publication, patent application or patent was specifically and individually indicated to be incorporated by reference in its entirety.

A number of embodiments of the invention have been described. Nevertheless, it will be understood that various modifications may be made without departing from the spirit and scope of the invention.

What is claimed is:

1. A method for isolating an antigen from an immune complex, comprising:
(a) applying a fluid sample to a first column that comprises an immunoglobulin-binding agent;
(b) collecting the flow-through eluant and detecting if immune complexes are present in the eluant;
(c) if immune complexes are present in the flow-through eluant of step (b), dissociating the immune complexes and isolating the antigen;
(d) if immune complexes are not present in the flow-through eluant of step
(b):
(d1) eluting the first column with an elution buffer that dissociates immune complexes;
(d2) applying the eluant of step (d1) to a second column comprising the immunoglobulin-binding agent;
(d3) collecting the flow-through eluant of step (d2).

2. The method of claim 1 wherein the immunoglobulin-binding agent is capable of binding IgG.

3. The method of claim 1, wherein the first or second column further comprises an agent capable of binding at least one molecule selected from the group consisting of albumin, transferrin, haptoglobin, antitrypsin, and fibrinogen.

4. The method of claim 1 wherein the first or second column comprises agents capable of binding IgG, IgA, albumin, transferrin, haptoglobin, and antitrypsin.

5. The method of claim 1 wherein the immunoglobulin-binding agent is an antibody.

6. The method of claim 1 wherein the fluid sample is serum or synovial fluid.

7. The method of claim 1 wherein the first or second column is a spin column.

8. The method of claim 1 wherein the fluid sample is obtained from a subject suffering from a disease or medical condition selected from the group consisting of rheumatoid arthritis, systemic lupus erythematosus, autoimmune pancreatitis, hepatitis B, Lyme disease, pulmonary tuberculosis, lung cancer, leukemia, ovarian cancer and breast cancer.

9. A method for isolating an antigen from an immune complex, comprising:
(a) applying a fluid sample to a first column that comprises an IgG-binding agent under conditions that allow the IgG-binding agent to bind the immune complex in the sample;
(b) eluting the first column with an elution buffer that dissociates immune complexes;
(c) applying the eluant of step (b) to a second column comprising the IgG-binding agent, wherein the second column further comprises an agent capable of binding at least one molecule selected from the group consisting of albumin, IgA, transferrin, haptoglobin, antitrypsin, and fibrinogen;
(d) collecting the flow-through eluant of step (c).

10. The method of claim 9 wherein the IgG-binding agent is an antibody.

11. The method of claim 9 wherein the first or second column is a spin column.

12. The method of claim 9 wherein the first or second column comprises an agent capable of binding albumin.

13. The method of claim 9 wherein the first or second column comprises agents capable of binding albumin, IgA, transferrin, haptoglobin, and antitrypsin.

14. The method of claim 9 wherein the fluid sample is serum or synovial fluid.

15. The method of claim 9 wherein the fluid sample is obtained from a subject suffering from a disease or medical condition selected from the group consisting of rheumatoid arthritis, systemic lupus erythematosus, autoimmune pancreatitis, hepatitis B, Lyme disease, pulmonary tuberculosis, lung cancer, leukemia, ovarian cancer and breast cancer.

16. A method for isolating an antigen from an immune complex, comprising:
(a) applying a fluid sample to a first column that comprises an IgG-binding agent and an agent capable of binding at least one molecule selected from the group consisting of albumin, IgA, transferrin, haptoglobin, antitrypsin, and fibrinogen, under conditions wherein the immune complex in the sample does not bind the IgG-binding agent in the column;
(b) collecting the flow-through from the first column and dissociate the immune complex;
(c) applying the eluant of step (b) to a second column comprising the IgG-binding agent and an agent capable of binding at least one molecule selected from the group consisting of albumin, IgA, transferrin, haptoglobin, antitrypsin, and fibrinogen, under conditions that allow IgGs to bind the IgG-binding agent;
(d) collecting the flow-through eluant of step (c).

17. The method of claim 16 wherein the IgG-binding agent is an antibody.

18. The method of claim 16 wherein the first or second column comprises agents capable of binding albumin, IgA, transferrin, haptoglobin, and antitrypsin.

19. The method of claim 16 wherein the fluid sample is serum or synovial fluid.

20. The method of claim 16 wherein the fluid sample is obtained from a subject suffering from a disease or medical condition selected from the group consisting of rheumatoid arthritis, systemic lupus erythematosus, autoimmune pancreatitis, hepatitis B, Lyme disease, pulmonary tuberculosis, lung cancer, leukemia, ovarian cancer and breast cancer.
